(19) United States
(12) Patent Application Publication
(10) Pub. No.: US 2020/0392055 A1
(43) Pub. Date: Dec. 17, 2020

(54) Improved Naphtha Steam Cracking Process

(71) Applicant: Total Research & Technology Feluy, Seneffe (BE)

(72) Inventors: Nikolai Nesterenko, Nivelles (Thines) (BE); Raoul Dethier, Schaerbeek (BE); Valérie Vanrysselberghe, Aalbeke (BE); Wolfgang Garcia, Braine-l'Alleud (BE)

(21) Appl. No.: 16/971,745

(22) PCT Filed: Feb. 21, 2019
(86) PCT No.: PCT/EP2019/054345

§ 371 Date: Aug. 21, 2020

(30) Foreign Application Priority Data
Feb. 22, 2018 (EP) 18158173.7
Feb. 22, 2018 (EP) 18158176.0

(51) Int. Cl. C07C 4/06; B01J 29/46; B01J 27/185; B01J 27/14

(52) U.S. Cl. CPC C07C 4/06; B01J 29/46; C07C 2527/185; B01J 27/14; C07C 2529/46; B01J 27/1853

(57) ABSTRACT

The invention relates to a process of catalytic conversion by dehydro steam cracking of paraffinic and naphthenic hydrocarbons from a naphtha feedstock to propylene in presence of steam, comprising the following steps:
a. providing a naphtha feedstock (1) containing one or more paraffins and/or naphthene's comprising 4 to 10 carbons atoms;
b. contacting (3) said naphtha feedstock (1) with a catalyst composition in the presence of steam in a reaction zone under dehydro steam cracking conditions at a temperature of at most 650° C., resulting in the production of an effluent (5);
c. recovering the effluent of step b) and separating (7) it into a converted fraction (9) and an unconverted fraction (11), wherein the unconverted fraction (11) comprises propane and one or more paraffins comprising 4 to 10 carbons atoms; and
d. submitting the unconverted fraction (11) to a steam cracking step;
   wherein the catalyst composition comprises one or more acid zeolite catalysts comprising at least one 10-membered ring channels, and one or more soft dehydrogenation elements containing basic compounds selected from rare-earth or alkaline earth metals oxide, salts or hydroxide.

### FIELD OF THE INVENTION

The invention relates to a process for producing propylene from a naphtha feedstock comprising paraffinic and naphthenic hydrocarbons. The invention also relates to the use of catalyst compositions comprising zeolites and basic compounds in a process for producing propylene from a naphtha feedstock.

### BACKGROUND OF THE INVENTION

The commercialization of shale gas and shale oil production via hydraulic fracturing combined with directional drilling (‘fracking’) have resulted in the production of natural gas liquids at costs and prices well below these of crude oil. One consequence of the price difference has been the aggressive announcement of new ethylene production capacity via steam cracking (primarily from ethane, but also combinations of propane and normal butane) in North America (where fracking originated), which was made in order to monetize the feedstock cost advantage when compared to conventional light naphtha steam cracking. Naphtha prices are closely correlated to crude oil, and currently several times higher than the prices of natural gas liquids. With the startup of additional ethane-based ethylene capacities (lighter fractions like ethane or liquefied petroleum gases (LPG) are considered very advantageous feedstock), the production of propylene and aromatics via the steam cracker declines.

World demand for propylene is expected to continually grow at an average annual rate of 4-5%. Currently, propylene is still mainly produced as a by-product of ethylene plants and Fluid catalytic cracking (FCC) units. Furthermore, the demand for propylene is actually growing faster than that of ethylene. To some extent, the propylene production can be optimized by transforming an FCC unit into a petrochemical FCC after a major revamp. However, a significant amount of dry gases and low values of by-products will be produced. In addition, an important revamp of the existing FCC unit and significantly higher process severity would be required to crack the naphtha to propylene and ethylene.

All the aforementioned factors have created an imbalance of supply and demand for propylene; a gap is being established between the available propylene supplies to meet the ongoing demand growth. While the markets have evolved to the point where modes of propylene by-product production can no longer satisfy the demand for propylene, the traditional classification of propylene as a “by-product” begun to evolve into more of classification as a “co-product” or even a “primary product”.

There is a need for the development of a more efficient technology to transform heavy feedstock (e.g. naphtha, atmospheric gas oil, whole crude oil, and hydrogenated vacuum gas oil) to propylene with significant yield advantages as compared to a classic naphtha steam cracker and petro FCC.

Many developments of the non-conventional thermal cracking processes include those that run on alternative feeds, such as crude oil and/or employ different reactor types, contact means and heat-supply methods.

In thermal cracking with partial combustion, ethylene and propylene can be made by cracking crude oil with heat supplied from superheated steam or hot gas generated by partial combustion in the upstream. The key features of this type of processes are high temperature, short residence time, and low hydrocarbon partial pressure. This type of technology includes the Advanced Cracking Reactor (ACR) process of Union Carbide, Kureha, and Chiyota, and Dow's partial combustion process.

Ethylene and propylene production using fluidized or circulating bed reactors is attractive because of its potential for overcoming coil metallurgy and heat flux restraints, abating coking problem, and processing low-cost heavy feedstocks. This type of process employs a direct contact heat transfer mechanism by using a solid heat carrier. Major processes of this type include Lurgi's sand cracker, BASF's fluidized flow cracking and coke cracking processes, the KK process developed by Kunugi and Kunii at the University of Tokyo, the Ube process, and Stone and Webster's Quick Contact Reaction System/Thermal Regenerative Process.

The Lurgi sand cracker uses fine-grained sand as the heat carrier. The sand is heated to about 850° C. (1562° F.) in a tubular sand lift by a fuel oil flame and is sent to a fluidized-bed reactor to heat and crack the feedstock at a temperature ranging from 730° C. to 850° C. (1346° F. to 1562° F.). The reaction time in the fluidized bed is between 0.3 and 0.6 seconds. The sand is continuously removed to the sand lift where coke is burned off, with the hot sand returned to the reactor. This process can crack feedstocks ranging from ethane to crude oil and has a flexible propylene/ethylene weight ratio varying from 0.3 to 0.9. The ethylene and propylene yields are 23.8 and 10.2 wt %, respectively, when cracking a crude oil.

The drawbacks of the process are a mechanical attack from the circulating sand on the refractory lining, high energy consumption, excessive solids attrition, and reactor instability. The Lurgi sand cracker was first used for ethylene production around 1958, and commercial plants were operated using various feedstocks in the 1960s but are no longer in operation.

BASF Fluidized Coke Cracking uses coke particles as the heat carrier. A mixture of pre-heated steam and O₂ is blown in under the reactor grid. The preheated crude oil and the recovered heavy oil are introduced into the fluidized bed, which is kept at a temperature ranging between 700° C. to 750° C. (1292° F. to 1382° F.). The cracking of crude oils yields about 20.6 to 23.0 wt % of ethylene and about 11.6 to 12.5 wt % of propylene.

A variation of this process is BASF's Fluidized Flow Cracking in which a regenerator is used to burn excess coke. The heat of combustion in the regenerator is carried to the reactor by ceramic solids. The cracking temperature in the Fluidized Flow Cracking is higher than in Fluidized Coke Cracking, and the ethylene yield is also higher. The cracking of same feedstocks, as reported for the Fluidized Coke Cracking, yields about 22.0 to 25.0 wt % of ethylene and about 10.5 to 11.3 wt % of propylene. BASF's Fluidized Coke Cracking was in commercial operation in the early 1960s and BASF's Fluidized Flow Cracking began operations in 1970. Neither is currently in operation.

The KK process uses coke particles as the heat carrier. Coke particles heated to about 1000° C. (1830° F.) by partial combustion and preheated crude are introduced to a fluidized-bed reactor. Cracking occurs at a temperature ranging from 700° C. to 800° C. (1292° F. to 1616° F.) with a residence time of about 0.5 to 0.8 second. The coke formed by the cracking is usually more than enough to make up for the coke consumed. The cracking of an Arabian Light crude oil yields ethylene, propylene, and coke at 20.9, 10.8, and 3.3 wt %, respectively.

Because coke has a low heat capacity, a large amount of coke in circulation is required to maintain the desired cracking temperature. Higher olefin yields are hard to achieve because it is difficult to operate the process at a higher temperature and lower residence time. Moreover, it is difficult to handle the carryover of fine coke particles into the downstream separation facility. The KK process was demonstrated in pilot plants run at different stages in 1980.

The Ube process uses inorganic oxide particles (mullite) as the heat carrier. Heat is supplied by partial combustion of crude oil. Steam and O₂, as the fluidizing gas, are supplied to the bed through a distributor. Simultaneously, steam is injected, as a jet stream, directly into the bed through a pipe at the centre of the distributor to maintain the solid particles in a forced-circulation flow within the cracking chamber. Preheated crude is fed along with the jet stream into the bottom of the fluidized bed. Cracking occurs at the temperature attained by partial combustion ranging from 830° C. to 880° C. (1526° F. to 1616° F.) with a residence time of about 0.2 to 0.3 second. A continuous regeneration system removes coke from the particles. Ube's process operates at a higher cracking temperature than do BASF's and the KK processes. It has a higher ethylene yield of about 28.1 to 34.6 wt %, but a lower propylene yield of about 6.3 to 13.8 wt %. The Ube process was tested in a demonstration plant in 1979.

The Quick Contact (QC) Reaction System is an outgrowth of Thermal Regeneration Cracking, which was developed by Stone and Webster and Gulf Oil Products (now a part of Chevron) based on Gulf's FCC riser reactor technology. It is not a specific process for olefin production; instead, possible applications include situations when short-residence-time, heterogeneous, residue-forming reactions occur. Depending on the nature of the reactions involved, either inert or catalytic solids can be used as the heat carrier. QC can process a broad range of feedstocks, including light alkanes, naphtha, diesel, vacuum gasoil, and residual oil.

In operation, a steam-diluted feedstock is partially or fully vaporized before it comes in contact with a fluidized bed or recirculating hot solids, on which thermal cracking reactions take place immediately. The cracking reactions are followed by gas-solids separation and water quench of the cracked gas. The solids are sent to a companion regenerator to be reheated with low-grade fuels and recirculated to the reactor. Carbon deposits on the solids are burned off during regeneration. With a steam/feedstock weight ratio of 0.2:1.0, the contact time is 0.1 to 0.25 second at a temperature ranging from 800° C. to 1000° C. (1472° F. to 1832° F.). When cracking a vacuum gasoil, the ethylene and propylene yields are 23.5 and 5 wt %, respectively.

QC has the following advantages: short residence time and high temperatures, minimum back mixing, no fouling, extreme feedstock flexibility, good heat input/removal and temperature uniformity, minimum pressure drop, high reactor capacity, continuous reaction and regeneration, and higher radiant thermal efficiency. However, QC has limitations: It is not applicable for reactions lasting longer than 2 seconds; it has a low catalyst loading capacity and solids attrition, and its investment cost is relatively high.

This process was demonstrated from 1979 to 1983, and detailed mechanical design was completed in 1989. The focus for QC has been on potential applications in FCC because a suitable catalyst for olefin production has yet to be found.

A recent development has been direct crude oil steam cracking. In January 2014 ExxonMobil officially opened in Singapore a novel steam cracker that produces olefins directly from crude oil. The Saudi Arabian Oil Company (Aramco) and Sabic have discussed plans to build a crude-to-olefins complex. However, a preconditioning and purification process is necessary to process these kinds of feedstock.

An example of process for production of ethylene and propylene by catalytic pyrolysis of heavy hydrocarbons is given by EP0909804. The heavy hydrocarbons are contacted with a pillared interlayered clay molecular sieve and/or phosphorus and aluminium or magnesium or calcium modified high silica zeolite having a structure of pentasil contained catalysts, in a riser or downflow transfer line reactor, in the presence of steam, and catalytically pyrolysed at a temperature ranging from 650° C. to 750° C. and a pressure ranging from 0.15 to 0.4 MPa for a contact time of about 0.2 to 5 seconds. The weight ratio of catalyst to feedstock is of 15:1 to 40:1 and the weight ratio of steam to feedstock is of 0.3:1 to 1:1. The yields of ethylene and propylene are over 18 wt %.

Additionally US 2014/275673 describes a process for producing light olefins and aromatics by contacting the feedstock with a catalytic cracking catalyst in at least two reaction zones. This process produces light olefins and BTX from heavy feedstocks.

US 2007/083071 discloses a process for increasing production of light olefinic hydrocarbons by catalytic cracking followed by thermal cracking.

U.S. Pat. No. 5,523,502 discloses an olefin production process with a deep catalytic cracking process together with a steam cracking process.

In order to improve the yield of propylene from heavy feedstock and be processing the feedstock with contaminants, a combination of low temperature catalytic cracking step with high temperature non-catalytic is advantageous. Low temperature shapes selective to propylene catalyst will maximize the yield of propylene from easily crackable part of the feedstock, decompose impurities (oxygenates, nitrides, sulfides) and at least partially extracts the metals from the feedstock. Then, the effluent may be significantly easily separated and a partially cracked at the classic conversion zone.

### SUMMARY OF THE INVENTION

The invention proposes a solution to significantly increase the production of propylene and BTX products (Benzene, Toluene, Xylene) from naphtha, to reduce expensive purification steps to remove oxygenates and to decrease the production of methane. The solution consists in an implementation of a catalytic pre-cracking process to transform a part of the easily crackable C4-C10 hydrocarbons to propylene and to decompose the contaminants. The fraction of C1 will contain only a very little amount of H₂, CH₄, and C2 and should not be necessary separated. All the contaminants like CO₂, CO etc will be removed with this fraction. The fact that the pre-cracking will occur at low temperature on a shape-selective catalyst will favour a production of propylene vs ethylene.

It has been found in accordance with the present invention that the use of a catalytic dehydro-cracking step at a temperature below 650° C., as a feed pre-treatment for the steam cracking for naphtha feedstock, allows the propylene/ethylene weight ratio in the steam cracking effluent to be shifted to values higher than the normal ranges observed when a straight run of naphtha is subject to a direct steam cracking. In an existing steam cracking plant, the throughput of the steam cracking furnace has a maximum value. Thus, the present invention provides the advantage that using the steam cracker, and without increasing the maximum throughput permitted by said steam cracker, the propylene yield may be dramatically increased by changing the feedstocks for the steam cracker. In particular, the propylene yield may be increased by using a catalytic dehydro cracking of paraffins as a pre-treatment for at least part of the steam cracking, with additional pre-fractionation of the effluent from the olefinic cracking process so as to remove propylene and ethylene primarily from the feedstock for the steam cracker.

According to a first aspect, the invention provides a process of catalytic conversion by dehydro steam cracking of paraffinic and naphthenic hydrocarbons from a naphtha feedstock to propylene in presence of steam, the process being remarkable in that it comprises the following steps:
a. providing a naphtha feedstock being a naphtha boiling range feedstock containing one or more paraffins and/or naphthene's comprising 4 to 10 carbons atoms;
b. contacting said naphtha feedstock with a catalyst composition in the presence of steam in a reaction zone under dehydro steam cracking conditions at a temperature of at most 650° C., resulting in the production of an effluent;
c. recovering the effluent of step b) and separating it into a converted fraction and an unconverted fraction, wherein the converted fraction comprises ethylene, propylene and BTX products, and wherein the unconverted fraction comprises propane and one or more paraffins comprising 4 to 10 carbons atoms; and
d. submitting the unconverted fraction to a steam cracking step, under steam cracking conditions;
   and in that the catalyst composition comprises one or more acid zeolite catalysts comprising at least one 10-membered ring channels, and one or more soft dehydrogenation elements containing basic compounds selected from rare-earth or alkaline earth metals oxide, salts or hydroxide.

Surprisingly, it was found by the inventors that a catalyst composition comprising a dehydrogenation catalyst combined with acid materials allows performing dehydrogenation and cracking reaction (DCN reaction) of a part of the naphtha to propylene with a consecutive conventional steam cracking reaction (SC reaction) on the propane and C4+ fraction recovered. The combination of DCN and SC processes leads to an increase of the propylene yield as compared to the conventional SC reaction performed. It was also found that the inventive process offered more flexibility and lead to higher propylene and ethylene ratio, low methane production and BTX (benzene, toluene, and xylenes) yield as compared to naphtha steam cracker and petro FCC. In case of the presence of contaminants in the naphtha feedstock, the first step will remove the contaminants and allows the direct processing of the non-converted fraction in the second reaction zone of steam cracking. The second soft dehydrogenation function also plays a role of a metal trap. This function allows protecting the cracking function of zeolite and avoid deterioration of its activity.

It is noted that WO2012/059191 describes a process for producing lower olefins from an oxygenated feedstream, using a catalyst composition comprising M1-M2-P/ZSM-5, wherein M1 is one or more basic compounds, M2 is one or more redox elements selected from Groups 6-8 of the Periodic Table of Elements and Sn and P is phosphorus, wherein said basic compound is a molecular entity forming a weak Lewis base and/or a weak Bronsted base in the catalyst composition. The feedstream used in this process comprises an oxygenate which is selected from the group consisting of dimethyl ether (DME), diethyl ether, methanol (MeOH) and ethanol (EtOH) or a mixture thereof. This document is silent about the possible use of said catalyst on a naphtha feedstock.

With preference, one or more of the following embodiments can be used to better define the inventive process:
The olefin content in the naphtha feedstock is less than 5 wt % of the total weight of said naphtha feedstock. Preferably, the naphtha feedstock is free of olefins.
In a preferred embodiment, the naphtha feedstock is a straight run naphtha, i.e. a naphtha obtained directly at the exit of the atmospheric distillation or a crude oil.
The dehydro steam cracking conditions comprise the naphtha feedstock being contacted with the catalyst at a temperature ranging from 500° C. to 650° C., preferably ranging from 530° C. to 630° C., more preferably ranging from 550° C. to 600° C.
The dehydro steam cracking conditions comprise the naphtha feedstock being contacted with the steam and the catalyst composition at a pressure ranging from 0.05 to 1.00 MPa, preferably in the range of 0.10 to 0.50 MPa.
The dehydro steam cracking conditions comprise the naphtha feedstock being contacted with the catalyst at a WHSV (feed) of at least 0.1 h⁻¹, preferably at a WHSV (feed) ranging from 0.1 h⁻¹ to 10.0 h⁻¹, more preferably from 0.5 h⁻¹ to 8.0 h⁻¹, even more preferably from 1.0 h⁻¹ to 6.0 h⁻¹, and most preferably from 1.5 h⁻¹ to 5.0 h⁻¹ preferably in a fixed bed reactor.
The dehydro steam cracking conditions comprise the naphtha feedstock being contacted with the steam and the catalyst composition at a naphtha feedstock partial pressure of at most 0.2 MPa.
The catalyst composition comprises a weight ratio between the basic and acid elements being the acid zeolite catalysts in the range from 1:5 to 5:1.
Steam is provided to the naphtha feedstock at a weight ratio steam/naphtha ranging from 1:10 to 10:1.

With preference, one or more of the following embodiments can be used to better define the steam cracking conditions of step d):
Steam is provided to the unconverted fraction at a weight ratio steam/naphtha ranging from 0.2 to 0.5 kg of steam per kg of the unconverted fraction.
The steam cracking step d) is performed at an outlet coil temperature of from 760° C. to 860° C.
The steam cracking step d) is performed at a pressure ranging from 0.07 MPa to 0.1 MPa.

With preference, one or more of the following embodiments can be used to better define the catalyst composition:
The catalyst composition is provided as a mixture of the acid zeolite catalysts and the soft dehydrogenation elements, or the acid zeolite catalysts and the soft dehydrogenation elements are provided separately.
The one or more basic compounds are selected from MgO, CaO, SrO, BaO, BeO, CeO₂, La₂O₃ and any mixture thereof, preferably is MgO or MgO—CeO₂.
The soft dehydrogenation elements are free of noble metals and contain essentially basic compounds and binder. The one or more basic compounds are selected from MgO, CaO, SrO, BaO, BeO, CeO₂, La₂O₃ and any mixture thereof, preferably is MgO or MgO—CeO₂. The content of noble metals is less than 500 ppm and of transition metals is less than 1.0 wt % as based on the total weight of the soft dehydrogenation elements, preferably the content of noble metals is less than 200 ppm, transition elements is less than 0.1 wt %. The impurities of transition elements may be present as a component of clays, which are used as a possible binder for catalyst formulation.
The catalyst composition comprises at least 0.5 wt % of the one or more basic compounds as based on the total weight of the catalyst composition, preferably at least 1.0 wt %, and more preferably at least 2.0 wt %.
The catalyst composition comprises at most 60 wt % of the one or more basic compounds as based on the total weight of the catalyst composition, preferably at most 40 wt % and more preferably at most 30 wt %.
The catalyst composition comprises one or more acid zeolite catalysts selected from the list comprising ZSM-5, silicalite-1, boralite C, TS-1, ZSM-11, silicalite-2, boralite D, TS-2, SSZ-46, MCM-68, CIT-1, SSZ-33, ZSM-8, Ferrierite, FU-9, ZSM-35, ZSM-23, ZSM-22, Theta-1, NU-10, ZSM-50, EU-1, ZSM-57, SAPO-11 and ZSM-48.
The catalyst composition comprises an acid zeolite catalyst of the MFI-type, preferably the catalyst composition comprises ZSM-5.
More than 20 wt % of the one or more acid zeolite catalysts of the catalyst composition are phosphorus treated acid zeolite catalysts, as based on the total weight of the acid zeolite catalysts in the catalyst composition, with preference catalyst composition comprises P/ZSM-5, i.e. ZSM-5 modified with phosphorous.
In an embodiment, the phosphorous modified acid zeolite catalyst is further modified to introduce at least 0.1 wt % of Mg, Ca, Sr, Ba, Ce, La, Fe, Ga. The concentration of the metal on the zeolite is preferably at most 5 wt %.
In an embodiment, the acid zeolite catalyst may be steamed before and after phosphorous and metal introduction at a temperature between 500° C.−750° C. for a period from 0.1 to 24 h under steam pressure from 0.1 to 10 bars.
The catalyst composition comprises at least 0.1 wt % of phosphorus as based on the total weight of the phosphorus treated acid zeolite catalyst, preferably at least 0.5 wt %, and more preferably at least 1.0 wt %.
The catalyst composition comprises at most 10 wt % of phosphorus as based on the total weight of the phosphorus treated acid zeolite catalyst, preferably at most 7.0 wt % more preferably at most 5.0 wt % and even more preferably at most 4.0 wt %.
The one or more acid zeolite catalysts have a framework Si/AI molar ratio of at least 10, preferably ranging from 10 to 100, more preferably ranging from 30 to 80.
The one or more acid zeolite catalysts are alkali metal-free. The content of alkali metal is less than 1.0 wt % as based on the total weight of the acid zeolite catalyst, preferably less than 0.1 wt %.
The soft dehydrogenation elements and acid zeolite catalyst is a part of the same or different catalyst particles.
The catalyst composition further comprises a binder selected from silica, alumina, clays, alumina phosphates, mullite, zirconia, titania, yttria; preferably the binder is silica, alumina, clays and alumina phosphates.

According to a second aspect, the invention provides the use of a catalyst composition in a catalytic conversion by dehydro steam cracking of paraffinic and naphthenic hydrocarbons from a naphtha feedstock to propylene in the presence of steam, wherein the process is as defined according to the first aspect, and is remarkable in that the catalyst composition comprises one or more acid zeolite catalysts comprising at least one 10-membered ring channels, and one or more soft dehydrogenation elements containing basic compounds selected from rare-earth or alkaline earth metals oxide, salts or hydroxide.

### DESCRIPTION OF THE FIGURES

FIG. 1 illustrates the process according to the invention.

### DETAILED DESCRIPTION OF THE INVENTION

For the purpose of the invention, the following definitions are given:

Naphtha is mainly a mixture of straight-chain, branched and cyclic aliphatic hydrocarbons. Naphtha is generally divided into light naphtha having from 4 to 10 carbon atoms per molecule and heavy naphtha having from 7 to 12 carbons per molecule. Typically, light naphtha contains naphthenes, such as cyclohexane and methyl-cyclopentane, and linear and branched paraffins or alkanes, such as hexane and pentane. Light naphtha typically contains from 60% to 99% by weight of paraffins and cycloparaffins.

The terms “alkane” or “alkanes” are used herein to describe acyclic branched or unbranched hydrocarbons having the general formula CₙH₂ₙ₊₂, and therefore consisting entirely of hydrogen atoms and saturated carbon atoms; see e.g. IUPAC. Compendium of Chemical Terminology, 2nd ed. (1997). Accordingly, the term “alkanes” describes unbranched alkanes (“normal-paraffins” or “n-paraffins” or “n-alkanes”) and branched alkanes (“iso-paraffins” or “iso-alkanes”) but excludes naphthene's (cycloalkanes).

The terms “aromatic hydrocarbons” or “aromatics” relate to cyclically conjugated hydrocarbon with a stability (due to electron delocalisation) that is significantly greater than that of a hypothetical localized structure (e.g. Kekule structure). The most common method for determining aromaticity of a given hydrocarbon is the observation of diatropicity in the ¹H NMR spectrum.

The terms “naphthenic hydrocarbons” or “naphthenes” or “cycloalkanes” are used herein describes saturated cyclic hydrocarbons.

The term “olefin” as used herein relates to an unsaturated hydrocarbon compound containing at least one carbon-carbon double bond. Preferably, the term “olefins” relates to a mixture comprising two or more compounds selected from of ethylene, propylene, butadiene, butylene-1, isobutylene, isoprene, and cyclopentadiene.

The term “LPG” as used herein refers to the well-established acronym for the term “liquefied petroleum gas”. LPG as used herein generally consists of a blend of C2-C4 hydrocarbons i.e. a mixture of C2, C3, and C4 hydrocarbons.

One of the petrochemical products which may be produced in the process of the present invention is BTX. The term “BTX” as used herein relates to a mixture of benzene, toluene, and xylenes.

As used herein, the term “C # hydrocarbons”, wherein “#” is a positive integer, is meant to describe all hydrocarbons having # carbon atoms. C # hydrocarbons are sometimes indicated as just “C #”. Moreover, the term “C #+ hydrocarbons” is meant to describe all hydrocarbon molecules having # or more carbon atoms. For instance, the term “C5+ hydrocarbons” is meant to describe a mixture of hydrocarbons having 5 or more carbon atoms. Furthermore, the term “C5+ alkanes” relates to alkanes having 5 or more carbon atoms.

The term “zeolite” refers to a molecular sieve aluminosilicate material. Reference herein to a zeolite having acid 10-membered ring channels is to a zeolite or aluminosilicate having 10-membered ring channels in one direction, optionally intersected with 8, 9 or 10-membered ring channels in another direction.

The term “redox element” as used herein relates to an element that forms different oxides with at least two different valencies and which can easily change from one valence to another one.

The alkaline earth metals (or Group 2 elements of the Periodic Table of Elements) which preferably may be comprised in the catalyst composition are selected from the group consisting of beryllium (Be), magnesium (Mg), calcium (Ca), strontium (Sr), barium (Ba) and radium (Ra), and more preferably selected from the group consisting of Mg, Ca and Sr.

The alkali metals represent the group in the periodic table consisting of the chemical elements lithium (Li), sodium (Na), potassium (K), rubidium (Rb), caesium (Cs), and francium (Fr).

A rare-earth element (REE) or rare-earth metal (REM), as defined by IUPAC, is one of a set of seventeen chemical elements in the periodic table, specifically the fifteen lanthanides, as well as scandium and yttrium. Scandium and yttrium are considered rare-earth elements because they tend to occur in the same ore deposits as the lanthanides and exhibit similar chemical properties. Rare-earth elements are cerium (Ce), dysprosium (Dy), erbium (Er), europium (Eu), gadolinium (Gd), holmium (Ho), lanthanum (La), lutetium (Lu), neodymium (Nd), praseodymium (Pr), promethium (Pm), samarium (Sm), scandium (Sc), terbium (Tb), thulium (Tm), ytterbium (Yb) and yttrium (Y).

The term “basic compound” relates to substances that, in aqueous solution react with acids to form salts, accept protons from any proton donor, and/or contain completely or partially displaceable OH⁻ ions.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The recitation of numerical ranges by endpoints includes all integer numbers and, where appropriate, fractions subsumed within that range (e.g. 1 to 5 can include 1, 2, 3, 4 when referring to, for example, a number of elements, and can also include 1.5, 2, 2.75 and 3.80, when referring to, for example, measurements). The recitation of endpoints also includes the recited endpoint values themselves (e.g. from 1.0 to 5.0 includes both 1.0 and 5.0). Any numerical range recited herein is intended to include all sub-ranges subsumed therein.

The particular features, structures, characteristics or embodiments may be combined in any suitable manner, as would be apparent to a person skilled in the art from this disclosure, in one or more embodiments.

The process of the invention provides an improved yield in propylene production as compared to ethylene production.

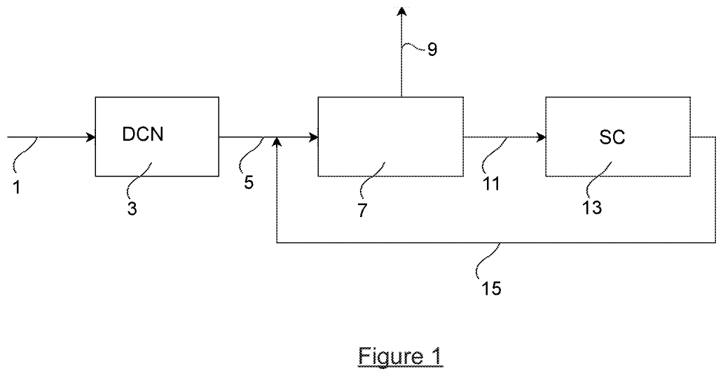

Reference is made to FIG. 1. The invention provides a process of catalytic conversion by dehydro steam cracking of paraffinic and naphthenic hydrocarbons from a naphtha feedstock to propylene in presence of steam, the process being remarkable in that it comprises the following steps:
a. providing a naphtha feedstock 1 being a naphtha boiling range feedstock containing one or more paraffins and/or naphthene's comprising 4 to 10 carbons atoms;
b. contacting 3 said naphtha feedstock 1 with a catalyst composition in the presence of steam in a reaction zone under dehydro steam cracking conditions at a temperature of at most 650° C., resulting in the production of an effluent 5;
c. recovering the effluent of step b) and separating 7 it into a converted fraction 9 and an unconverted fraction 11, wherein the converted fraction comprises ethylene, propylene and BTX products and wherein the unconverted fraction comprises propane and one or more paraffins comprising 4 to 10 carbons atoms; and
d. submitting the unconverted fraction to a steam cracking step 13, under steam cracking conditions;
   and in that the catalyst composition comprises one or more acid zeolite catalysts comprising at least one 10-membered ring channels, and one or more soft dehydrogenation elements containing basic compounds selected from rare-earth or alkaline earth metals oxide, salts or hydroxide.

In an embodiment, the effluent 15 recovered after the steam cracking step is further separated into a converted fraction and an unconverted fraction, wherein the converted fraction comprises ethylene, propylene and BTX products and wherein the unconverted fraction comprises propane and one or more paraffins comprising 4 to 10 carbons atoms. In a preferred embodiment, the effluent 15 is mixed with effluent 5 before the separation step 7.

The naphtha feedstock used in the invention comprises paraffinic and naphthenic hydrocarbons, preferably the naphtha feedstock comprises one or more paraffins comprising 4 to 10 carbon atoms.

The naphtha feedstock may comprise compounds other than paraffins. Preferably, the naphtha feedstock comprises at least 10 wt % of paraffins comprising 4 to 10 carbon atoms as based on the total weight of the naphtha feedstock, more preferably at least 50 wt %, and more preferably at least 60 wt % of paraffins comprising 4 to 10 carbon atoms.

Preferably, the naphtha feedstock comprises from 10 wt % to 100 wt % of paraffins comprising 4 to 10 carbon atoms as based on the total weight of the naphtha feedstock, more preferably from 50 wt % to 99.5 wt %, and more preferably from 60 wt % to 95 wt % of paraffins comprising 4 to 10 carbon atoms.

The naphtha feedstock may comprise straight run naphtha or naphtha fractions derived from natural gas, natural gas liquids or associated gas. The feedstock may comprise naphtha fractions derived from pyrolysis gas. The naphtha feedstock may also comprise naphtha or naphtha fractions obtained from a Fischer-Tropsch process for synthesising hydrocarbons from hydrogen and carbon monoxide. For example, the naphtha feedstock is or comprises desalted light crude oil and shale oil.

The naphtha feedstock may also comprise higher paraffins, i.e. paraffins comprising more than 10 carbon atoms. Cracking such higher paraffins typically requires the use of temperatures and pressures which are at the higher end of the preferred temperature and pressure ranges.

Preferably, the naphtha feedstock comprises at least 10% of naphthene's. More preferably, the naphtha feedstock comprises in the range of from 10 to 40 wt %, more preferably of from 50 to 90 wt % of naphthene's and paraffins C6+, based on the total weight of the naphtha feedstock.

The naphtha feedstock may comprise olefins. However, as the olefins are significantly more reactive during the dehydro steam cracking process relative to the naphthene's and paraffins, the presence of olefins may result in a fast coking of catalyst and would require to regenerate it too frequently. Preferably, the naphtha feedstock may comprise from 0 to 20 wt % of olefins and preferably less than 0.2 wt % of diolefins, based on the total weight of the naphtha feedstock. More preferably, the naphtha feedstock may comprise from 0 to 10 wt % of olefins and less than 0.1 wt % diolefins. Optionally, the naphtha feedstock is subjected to a selective hydrogenation treatment prior to being supplied to a process according to the present invention.

The zeolite having acid 10-membered ring channels that can be used for the invention can be selected from:
one or two dimensional zeolites having 10-membered ring channels in one direction, which are not intersected by others channels from other directions;
three-dimensional zeolites having intersecting channels in at least two directions, whereby the channels in one direction are 10-membered ring channels, intersected by 8, 9 or 10-membered ring channels in another direction.

Examples of 10-membered ring channels zeolites suitable for the process of the invention can be of, but not limited to, the MFI-type, the MEL-type, the MSE-type, the CON-type, the ZSM-8-type, the FER-type, the MTT-type, the TON-type, the EUO-type, the MFS-type, the AEL-type and the ZSM-48-type zeolites. Preferably, the catalyst is or comprises a zeolite of the MFI-type.

MFI-type zeolites have a three-dimensional structure. Preferably, the zeolite of the MFI-type is selected from ZSM-5, silicalite-1, boralite C, and TS-1. The preferred MFI-type zeolite is ZSM-5. MEL-type zeolites have a three-dimensional structure. Preferably, the zeolite of the MEL-type is selected from ZSM-11, silicalite-2, boralite D, TS-2, and SSZ-46. The preferred zeolite of the MSE-type is MCM-68. The zeolite of the CON-type is selected from CIT-1 and SSZ-33. The zeolite of the FER-type is selected from Ferrierite, FU-9 and ZSM-35. The preferred zeolite of the MTT-type is ZSM-23. The zeolite of the TON-type is selected from ZSM-22, Theta-1 and NU-10. The zeolite of the EUO-type is selected from ZSM-50 and EU-1. The preferred zeolite of the MFS-type is ZSM-57. The preferred zeolite of the AEL-type is SAPO-11. ZSM-48 refers to the family of microporous materials consisting of silicon, aluminium, oxygen and optionally boron.

Preferably, the catalyst comprises one or more zeolites selected from the list comprising ZSM-5, silicalite-1, boralite C, TS-1, ZSM-11, silicalite-2, boralite D, TS-2, SSZ-46, MCM-68, CIT-1, SSZ-33, ZSM-8, Ferrierite, FU-9, ZSM-35, ZSM-23, ZSM-22, Theta-1, NU-10, ZSM-50, EU-1, ZSM-57, SAPO-11 and ZSM-48. More preferably, the catalyst is or comprises ZSM-5 zeolite.

Preferably, more than 20 wt % of the one or more acid zeolite catalysts of the catalyst composition are phosphorus treated acid zeolite catalysts, as based on the total weight of the acid zeolite catalysts in the catalyst composition, preferably more than 50 wt %, more preferably more than 80 wt %, even more preferably more than 90 wt %, and most preferably 100 wt % of the zeolites catalysts are phosphorus treated acid zeolite catalysts. With preference, the catalyst composition comprises P/ZSM-5.

In an embodiment, the phosphorous modified acid zeolite catalyst is further modified to introduce at least 0.1 wt % of Mg, Ca, Sr, Ba, Ce, La, Fe, Ga. The concentration of the metal on the zeolite is preferably at most 5 wt %. With preference, the catalyst composition comprises Fe-P/ZSM-5 and/or Ca— P/ZSM-5. More preferably, the catalyst composition comprises Fe-P/ZSM-5.

In an embodiment, the acid zeolite catalyst may be steamed before and after phosphorous and metal introduction at a temperature between 500° C.-750° C. for a period from 0.1 to 24 h under steam pressure from 0.1 to 10 bars.

The catalyst composition comprises at least 0.1 wt % of phosphorus as based on the total weight of the phosphorus treated acid zeolite catalyst, preferably at least 0.5 wt %, and more preferably at least 1.0 wt %.

The catalyst composition comprises at most 10 wt % of phosphorus as based on the total weight of the phosphorus treated acid zeolite catalyst, preferably at most 7.0 wt % more preferably at most 5.0 wt % and even more preferably at most 4.0 wt %.

The one or more acid zeolite catalysts have a framework Si/AI molar ratio of at least 10, preferably ranging from 10 to 100, more preferably ranging from 30 to 80. The one or more acid zeolite catalysts are alkali metal-free. The content of alkali metal is less than 1.0 wt % as based on the total weight of the acid zeolite catalyst, preferably less than 0.1 wt %.

The catalyst composition further comprises a binder selected from silica, alumina, clays, alumina phosphates, mullite, zirconia, titania, yttria; preferably the binder is silica, alumina, clays and alumina phosphates.

The catalyst composition of the present invention preferably comprises at least 10 wt % of a binder as based on the total weight of the catalyst composition, most preferably at least 20 wt % of a binder and preferably comprises up to 40 wt % of a binder.

In a further aspect, the catalyst composition used in the process of the present invention is prepared by the method comprising the steps of:
steaming acid zeolite catalyst at a temperature between 500° C.-750° C. for a period from 0.1 to 24 h under steam pressure from 0.1 to 10 bars.
contacting the steamed zeolite with the one or more source of phosphorus
optionally, introducing to the phosphate sample at least 0.1 wt % of Mg, Ca, Sr, Ba, Ce, La, Fe, Ga; The said metals may be further presented on catalyst in form of oxides, silicates, aluminates or phosphates;
drying and steaming of the one or more modified acid zeolite catalysts at a temperature between 500° C.−750° C. for a period from 0.1 to 24 h under steam pressure from 0.1 to 10 bars.

Accordingly, the one or more acid zeolite catalysts are contacted with a solution in which one or more basic compounds are dissolved, and wherein, with preference, one or more redox elements are dissolved as well. Preferably, the solution is an aqueous solution. Preferred source of phosphorous is the phosphoric acid. Preferred soluble salts of the basic compounds and of the redox elements are nitrate salts. Preferred soluble salts of the basic compounds are selected from the list consisting of Mg(NO₃)₂, Ca(NO₃)₂, Sr(NO₃)₂, La(NO₃)₃, Ga(NO₃)₃, Fe(NO₃)₃.

In an embodiment, he phosphorous modified acid zeolite catalyst is preferably obtained by the process described in WO2009/016156, which is incorporated herein by reference. The process comprises the following steps in this order:
selecting a zeolite with low Si/AI molar ratio (advantageously lower than 30) among H+ or NH₄⁺-form of MFI, MEL, FER, MOR, clinoptilolite, said zeolite having been made preferably without direct addition of organic template;
steaming at a temperature ranging from 400 to 870° C. for 0.01 to 200 h;
leaching with an aqueous acid solution containing the source of P at conditions effective to remove a substantial part of Al from the zeolite and to introduce at least 0.3 wt % of P;
separation of the solid from the liquid;
an optional washing step or an optional drying step or an optional drying step followed by a washing step;
a calcination step.

The basic compounds and the optional redox element(s) and phosphorus (P), may be deposited by contacting the one or more acid zeolite catalysts with a single solution in which the soluble salts of the basic compounds, soluble salts of the redox elements and phosphoric acid are dissolved.

Alternatively, the basic compounds and the optional redox element(s) and phosphorus (P) may be deposited by subsequently contacting the one or more acid zeolite catalysts with the different elements and/or phosphorus, whereby the composition is dried to evaporate the solvent before contacting the composition with the following element. After depositing all the required elements, the resulting composition (catalyst precursor) is dried.

In one embodiment of the present invention, the catalyst precursor is air-dried, preferably for about 8 hours at a temperature ranging from 60° C. to 80° C. while stirring.

After drying, the zeolite-comprising composition, on which the basic compounds and the optional redox element(s) and the phosphorus (P) are deposited, is calcined in an oxygen-comprising atmosphere, preferably in moisture-free atmospheric air. Preferably, the catalyst precursor is calcined at a temperature ranging from 450° C. to 550° C. to remove the residual amount of nitrates, and carbons.

Most preferably, the catalyst precursor is calcined at about 500° C. for about 4 hrs. When a binder is present, it is preferred that the one or more acid zeolite catalysts are mixed with the binder prior to contacting the one or more acid zeolite catalysts with one or more solutions comprising soluble salts of basic compounds and the optional soluble salts of redox elements and phosphoric acid.

In the process according to the invention, the naphtha feedstock is contacted with the catalyst at elevated temperature and low partial pressure of hydrocarbons pressure in dehydro steam cracking conditions.

In an embodiment, the dehydro steam cracking conditions comprise the naphtha feedstock being contacted with the catalyst at a temperature ranging from 500° C. to 650° C., preferably ranging from 500° C. to 630° C., more preferably ranging from 550° C. to 600° C.

Preferably, the dehydro steam cracking conditions comprise the naphtha feedstock being contacted with the steam and the catalyst composition at a pressure ranging from 0.05 to 1.00 MPa, preferably in the range of 0.10 to 0.50 MPa.

In an embodiment, the dehydro steam cracking conditions comprise the naphtha feedstock being contacted with the steam and the catalyst composition wherein the naphtha feedstock partial pressure is at most 0.2 MPa.

In an embodiment, the dehydro steam cracking conditions comprise the naphtha feedstock being contacted with the catalyst at a weight hourly space velocity of the naphtha feedstock (WHSV) of at least 0.1 h⁻¹, preferably is ranging from 0.1 h⁻¹ to 10.0 h⁻¹, more preferably from 0.5 h⁻¹ to 8.0 h⁻¹, even more preferably from 1.0 h⁻¹ to 6.0 h⁻¹, and most preferably from 1.5 h⁻¹ to 5.0 h⁻¹ preferably in a fixed bed reactor.

With preference, the steam cracking conditions of step d) comprise the steam being provided to the unconverted fraction at a weight ratio steam/naphtha ranging from 0.2 to 0.5 kg of steam per kg of the unconverted fraction.

In an embodiment, the steam cracking step d) is performed at an outlet coil temperature ranging from 760° C. to 860° C.

In an embodiment, the steam cracking step d) is performed at a pressure ranging from 0.07 MPa to 0.1 MPa

The steam cracking is a well-known process in which saturated hydrocarbons are broken down into smaller, often unsaturated, hydrocarbons. The process is described in the document WO2016/058953 incorporated herein by reference. In this process, the hydrocarbons are mixed with dilution steam before it flows into the heating zone where the temperature is of at least 820° C., the residence time ranges from 0.05 to 0.5 seconds, preferably from 0.1 to 0.4 seconds and the pressure ranges from 750 to 950 mbars, preferably from 800 to 900 mbars, more preferably being approximately 850 mbars.

### Methods

Gas chromatography was performed on Columns: DB1 (40 m, 0.1 mm, 0.4 μm) and Al₂O₃ (50 m, 0.32 mm, 5 μm) using Agilent operated by ChemStation software.

Phosphorus content is determined in accordance with UOP Method 961-12.

### EXAMPLES

The following examples illustrate the invention. In the examples, the yield of steam crackers was computer simulated using the SPYRO simulation software known in the art, in which a straight run naphtha or the fraction after the dehydro steam cracking was subjected to simulated steam cracking in a steam cracker.

### Example 1: Preparation of Fe-P/ZSM-5

A sample of zeolite ZSM-5 (Si/Al=11, CBV2314 Zeolyst) in NH₄-form (contained 250 ppm of Na and synthesized without template) was blended with a silica sol binder in a weight ratio 70:30 followed by addition of extrusion additives and shaping in form of cylinders of 1.8 mm in diameter. The extruded sample was dried for 2 h at 140° C., calcined for 2 h at 600° C. followed by steaming at 550° C. for 2 h in 100% steam.

Steamed solid was incipient wetness impregnated with an aqueous solution of phosphoric acid to introduce about 3 wt % of phosphorus to the catalyst. The impregnated solid was dried for 16 h at 110° C. Then, the dried solid was impregnated with Fe(NO₃)₃, 9H₂O to introduce 0.8 wt % of Fe on the catalyst

Resulted catalyst containing 2.8 wt % of phosphorus and 0.8% of calcium was steamed at 750° C. for 1 h in 100% of steam. The sample is hereinafter identified as catalyst A.

### Example 2: Preparation of Ca-P/ZSM-5

A sample of zeolite ZSM-5 (Si/Al=11, CBV2314 Zeolyst) in NH₄-form (contained 250 ppm of Na and synthesized without template) was blended with a 20 wt % of kaolin binder and 10 wt % of silica sol binder in a weight ratio zeolite/binder 70:30 followed by addition of extrusion additives and shaping in form of cylinders 1.8 mm in diameter.

The extruded sample was dried for 2 h at 140° C., calcined for 2 h at 600° C. followed by steaming at 550° C. for 2 h in 100% steam.

Steamed solid was incipient wetness impregnated with an aqueous solution of phosphoric acid to introduce about 3 wt % of phosphorus to the catalyst. The impregnated solid was dried for 16 h at 110° C.

Then the dried solid was impregnated with Ca(NO₃)₂ to introduce about 0.5 wt % of Ca on the catalyst.

Resulted catalyst containing 2.8 wt % of phosphorus and 0.4 wt % of calcium was steamed at 750° C. for 1 h in 100% of steam. The sample is hereinafter identified as catalyst B.

### Example 3

The process was conducted in a fixed bed reactor loaded with the catalyst A (Fe/P-ZSM-5)-containing catalyst blended 50:50 on weight basis with MgO/Al₂O₃ mixed oxide (30:70, MgO:Al₂O₃, Pural Mg30, Sasol). The demonstration of the invention was performed in micropilote. The zeolite is in its hydrogen form and the catalyst composition was extruded in cylinder form. MgO/Al₂O₃ mixed oxide is a soft dehydrogenation additive in the example.

A stainless-steel reactor tube having an internal diameter of 10 mm is used. 10 mL of the catalyst composition, as pellets of 35-45 mesh, is loaded in the tubular reactor. The void spaces, before and after the catalyst composition, are filled with SiC granulates of 2 mm. The temperature profile is monitored with the aid of a thermocouple well placed inside the reactor at the top of the catalyst bed. Before the reaction, the catalyst was activated at 575° C. for 6 h (heating rate 60° C./h) followed by sending steam to the catalyst with WHSV_{(H2O)} of 5 h⁻¹. After one hour-on-stream, naphtha feedstock was sent to the catalyst with WHSV₍ₙₐₚₕₜₕₐ₎ of 2.5 h⁻¹ (keeping steam injection). The performance test is performed down-flow at 1.5 barg of total pressure, weight ratio H₂O/Naphtha=1, WHSV₍ₙₐₚₕₜₕₐ₎=2.5 h⁻¹, Temperature=575° C. for 48 hours-on-stream. Analysis of the products is performed by using an on-line gas chromatography. The catalyst showed a stable performance.

The results are provided in table 1.

### Example 4

The process was conducted in a fixed bed reactor loaded with the catalyst B (Ca/P-ZSM-5)-containing catalyst blended 50:50 on weight basis with MgO/Al₂O₃ mixed oxide (30:70, MgO:Al2O3, Pural Mg30, Sasol). The demonstration of the invention was performed in micropilote. The zeolite is in its hydrogen form and the catalyst composition was extruded in cylinder form. MgO/Al₂O₃ mixed oxide is a soft dehydrogenation additive in the example.

A stainless-steel reactor tube having an internal diameter of 10 mm is used. 10 mL of the catalyst composition, as pellets of 35-45 mesh, is loaded in the tubular reactor. The void spaces, before and after the catalyst composition, are filled with SiC granulates of 2 mm. The temperature profile is monitored with the aid of a thermocouple well placed inside the reactor at the top of the catalyst bed. Before the reaction, the catalyst was activated at 575° C. for 6 h (heating rate 60° C./h) followed by sending steam to the catalyst with WHSV_{(H2O)} of 5 h⁻¹. After one hour-on-stream, naphtha feedstock was sent to the catalyst with WHSV₍ₙₐₚₕₜₕₐ₎ of 2.5 h⁻¹ (keeping steam injection). The performance test is performed down-flow at 1.5 barg of total pressure, weight ratio H₂O/Naphtha=1, WHSV₍ₙₐₚₕₜₕₐ₎=2.5 h⁻¹, Temperature=575° C. for 48 hours-on-stream. Analysis of the products is performed by using an on-line gas chromatography. The catalyst showed a stable performance.

The results are provided in table 1.

TABLE 1

| | Unit | FEED | Example 3 | Example 4 |
|---|---|---|---|---|
| methane | wt % | 0.0 | 1.1 | 1.0 |
| ethylene | wt % | 0.0 | 6.6 | 5.1 |
| ethane | wt % | 0.0 | 3.2 | 2.4 |
| propylene | wt % | 0.0 | 17.3 | 13.6 |
| propane | wt % | 0.0 | 2.7 | 3.1 |
| C4 | wt % | 9.0 | 14.9 | 7.1 |
| non-cyclic C5-C6 | wt % | 60.0 | 33.7 | 51.5 |
| non-cyclic C7-C8 | wt % | 6.0 | 5.2 | 5.1 |
| Naphthenes | wt % | 23.0 | 2.9 | 5.5 |
| BTX | wt % | 2.0 | 12.2 | 5.3 |
| C9+ | wt % | n.d. | <0.2 | <0.3 |

n.d.: not determined

The results show a weight ratio C3/C2 of about 3 on the products obtained, thus the production of propylene is favoured over ethylene. The yield of BTX obtained with catalyst A is high (12.2 wt %). The conversion of naphtenic hydrcarbons was between 75-90 wt %.

### Example 5

The mild hydrocracking of naphtha was followed by a separation section and a steam cracking section for the unconverted parts in order to produce propylene. The ultimate yield in DCN+SC was compared to a conventional steam cracker. The condition used were:
coil outlet temp 806.8° C.
inlet radiation temperature 535° C.
Steam dilution 0.30 kg/kg
Coil outlet pressure 2.15 Bara

The results are given in table 2.

TABLE 2

| | | SC | DCN + SC | |
|---|---|---|---|---|
| | | SPYRO | Example 3 + | Example 4 + |
| | | FEED | SPYRO | SPYRO |
| | Unit | stock | of C3-C6 | Of C3-C6 |
| CH4 | wt % | 13.4 | 8.3 | 9.9 |
| Other C2-C3 | wt % | 5.4 | 6.1 | 6.0 |
| Ethylene + | wt % | 43.4 | 47.4 | 47.9 |
| Propylene | | | | |
| C6-C8 Aromatics | wt % | 8.0 | 16.5 | 10.7 |
| (mainly BTX) | | | | |
| propylene | wt % | 18.6 | 27.4 | 26.1 |
| ethylene | wt % | 24.8 | 20.0 | 21.7 |
| C4 | wt % | 13.8 | 7.5 | 9.3 |
| C5-C8 | wt % | 14.4 | 13.0 | 14.8 |
| C9+ | wt % | 1.7 | 1.1 | 1.4 |

The results showed that the cumulated yield of ethylene and propylene was similar, but that propylene production is favoured by the process of the invention. In addition, the BTX yield is higher. Methane and heavy products production yields are significantly lowered.

### Example 6

The impact of the presence of the C4-C5 cut in the stream to be steamed cracked was also studied. The simulation performed in example 5 was performed on the stream obtained in example 3. The simulations were performed with similar operating conditions as in example 5. In a second simulation, the C4-C5 cut was removed in a similar way as in US2014/0275673 to determine the impact on the yields of this removal. The condition used were:
coil outlet temp 818° C.
inlet radiation temperature 535° C.
Steam dilution 0.30 kg/kg
Coil outlet pressure 2.15 Bara

The results are given in table 3.

TABLE 3

| | | SPYRO simulation | SPYRO simulation of the |
|---|---|---|---|
| | | of the C3-C6 of | C3-C6 of example 3 with |
| | | example 3 | the removal of C4-C5 |
| CH4 | wt % | 16.7 | 16.5 |
| Other C2-C3 | wt % | 5.7 | 6.0 |
| Ethylene + | wt % | 42.4 | 41.6 |
| Propylene | | | |
| C6-C8 Aromatics | wt % | 7.7 | 9.6 |
| (mainly BTX) | | | |
| propylene | wt % | 18.2 | 17.8 |
| ethylene | wt % | 24.2 | 23.8 |
| C4 | wt % | 13.5 | 13.1 |
| C5-C8 | wt % | 11.3 | 9.9 |
| C9+ | wt % | 1.8 | 2.4 |

The steam cracking is performed at a higher temperature than in examples 5, the yields are therefore slightly different. It appears that the removal of the C4-C5 cut leads to a higher production of heavies. In particular the C9+ are more abundant when the C4-C5 cut is removed. The heavies are known to be coke precursors. When more coke is formed, more often shutdowns of the steam cracking unit are required. It is therefore not particularly advantaging to remove the C4-C5 as it leads to more coking in the steam cracker tubes.

1.-15. (canceled)

16. A process of catalytic conversion by dehydro steam cracking of paraffinic and naphthenic hydrocarbons from a naphtha feedstock to propylene in presence of steam, the process being characterized in that it comprises the following steps:
a. providing a naphtha feedstock being a naphtha boiling range feedstock containing one or more paraffins and/or naphthene's comprising 4 to 10 carbons atoms;
b. contacting said naphtha feedstock with a catalyst composition in the presence of steam in a reaction zone under dehydro steam cracking conditions at a temperature of at most 650° C., resulting in the production of an effluent;
c. recovering the effluent of step b) and separating it into a converted fraction and an unconverted fraction, wherein the converted fraction comprises ethylene, propylene and BTX products and wherein the unconverted fraction comprises propane and one or more paraffins comprising 4 to 10 carbons atoms; and
d. submitting the unconverted fraction to a steam cracking step, under steam cracking conditions;
and in that the catalyst composition comprises one or more acid zeolite catalysts comprising at least one 10-membered ring channels, and one or more soft dehydrogenation elements containing basic compounds selected from rare-earth or alkaline earth metals oxide, salts or hydroxide.

17. The process according to claim 16 characterized in that the one or more basic compounds are selected from MgO, CaO, SrO, BaO, BeO, CeO₂, La₂O₃, and any mixture thereof.

18. The process according to claim 16, characterized in that the catalyst composition comprises:
at least 0.5 wt % of the one or more basic compounds as based on the total weight of the catalyst composition; and/or
at most 60 wt % of the one or more basic compounds as based on the total weight of the catalyst composition.

19. The process according to claim 16 characterized in that the catalyst composition comprises one or more acid zeolite catalyst selected from the list comprising ZSM-5, silicalite-1, boralite C, TS-1, ZSM-11, silicalite-2, boralite D, TS-2, SSZ-46, MCM-68, CIT-1, SSZ-33, ZSM-8, Ferrierite, FU-9, ZSM-35, ZSM-23, ZSM-22, Theta-1, NU-10, ZSM-50, EU-1, ZSM-57, SAPO-11 and ZSM-48.

20. The process according to claim 16, characterized in that more than 20 wt % of the one or more acid zeolite catalysts of the catalyst composition are phosphorus treated acid zeolite catalysts, as based on the total weight of the acid zeolite catalysts in the catalyst composition.

21. The process according to claim 20, characterized in that the catalyst composition comprises at least 0.1 wt % of phosphorus as based on the total weight of the phosphorus treated acid zeolite catalyst, and/or the catalyst composition comprises at most 10.0 wt % of phosphorus as based on the total weight of the phosphorus treated acid zeolite catalyst.

22. The process according to claim 16, characterized in that the one or more acid zeolite catalysts have a framework Si/Al molar ratio ranging from 10 to 100.

23. The process according to claim 16, characterized in that the dehydro steam cracking conditions comprise:
the naphtha feedstock being contacted with the catalyst at a temperature ranging from 500 to 650° C., and/or
the naphtha feedstock being contacted with the steam and the catalyst composition at a pressure ranging from 0.10 to 0.50 MPa.

24. The process according to claim 16, characterized in that the one or more acid zeolite catalysts are alkali metal-free.

25. The process according to claim 16, characterized in that the dehydro steam cracking conditions comprise the naphtha feedstock being contacted with the catalyst at a WHSV (feed) of at least 0.1 h⁻¹.

26. The process according to claim 16, characterized in that the dehydro steam cracking conditions comprise the naphtha feedstock being contacted with the steam and the catalyst composition at a naphtha feedstock partial pressure of at most 0.2 MPa.

27. The process according to claim 16 characterized in that steam is provided to the naphtha feedstock at a weight ratio steam/Naphtha ranging from 1:10 to 10:1.

28. The process according to claim 16, characterized in that the catalyst composition further comprises a binder selected from is selected from silica, alpha-alumina, gamma-alumina, clays, alumina phosphates, mullite, zirconia, titania, yttria, silicon nitride, silicon carbide, iron, bronze and stainless steel, glass, and carbon.

29. The process according to claim 16, characterized in that the catalyst composition comprises a weight ratio between the basic and acid elements being the acid zeolite catalysts in the range from 1:5 to 5:1.

30. The process according to claim 16, characterized in that the step d) is performed at an outlet coil temperature of from 760 to 860° C. and/or wherein steam is provided to the unconverted fraction at a weight ratio steam/unconverted fraction ranging from 0.2 to 0.5 kg of steam per kg of the unconverted fraction.
